(19) United States
(12) Patent Application Publication
(10) Pub. No.: US 2007/0148699 A1
(43) Pub. Date: Jun. 28, 2007

(54) METHOD FOR DERIVING A WELLNESS RELATED INDEX FOR A FOOD COMPOSITION

(76) Inventors: Kim Gene Friesen, Topeka, KS (US); Ryan Michael Yamka, Topeka, KS (US); Sonya Marie Woertz-Butler, Topeka, KS (US)

Correspondence Address: COLGATE-PALMOLIVE COMPANY, 909 RIVER ROAD, PISCATAWAY, NJ, 08855, US

(21) Appl. No.: 11/609,715

(22) Filed: Dec. 12, 2006

Related U.S. Application Data
(60) Provisional application No. 60/749,534, filed on Dec. 12, 2005.

(51) Int. Cl. G01N 33/53; G06F 19/00

(52) U.S. Cl. 435/7.1; 702/19

(57) ABSTRACT

A method for deriving a wellness related index for a composition for consumption by an animal by (a) evaluating an effect of the composition, relative to the effect of one or more comparative compositions, on each of a plurality of parameters indicative of the health or wellness of the animal; (b) expressing the relative effect of the composition on each parameter as a numerical score; and (c) computing the wellness related index using an algorithm that takes into account the numerical scores of the composition for the plurality of parameters.

### CROSS-REFERENCE TO RELATED APPLICATIONS

This application claims benefit of U.S. Provisional No. 60/749,534 filed Dec. 12, 2005 which is incorporated by reference in its entirety for all purposes.

### BACKGROUND OF THE INVENTION

1. Field of the Invention

This invention relates generally to methods for deriving wellness related indexes and particularly to methods for deriving a wellness related index for food compositions.

2. Description of the Related Art

An increasing number of people acquire and care for a great variety of pets. The American Pet Products Manufacturers Association estimates that in the year 2002 more than 64 million households in the United States had one or more pets, an increase of approximately 12 million since 1992. Many pet owners would like their pets to live longer and healthier lives. While pets and other animals today live longer and have a better quality of life due to improved nutrition and medical care, animal care-givers still need a method for determining the suitability of foods for animals with diverse needs. Many foods are available to choose from and it is difficult for animal care-givers such as veterinarians, farmers and pet owners to choose the best food for a particular animal or group of animals.

There are many indicators of health and wellness in animals, including markers such as biomarkers, behavioral indicators, biometrics, etc. It would be of value to evaluate a plurality of health and wellness indicators and provide from that cumulative information a way of describing the health and wellness of an animal, or an index that relates to the health and wellness of an animal. More particularly, it would be helpful to provide an index for a food that would give an animal care-giver an easily visualized method of determining the food's suitability for an animal, and would indicate the food's effect on health and wellness in that animal.

### SUMMARY OF THE INVENTION

There is provided a method for deriving a wellness related index for a composition for consumption by an animal. The method comprises evaluating an effect of the composition, relative to the effect of one or more comparative compositions, on each of a plurality of parameters indicative of the health or wellness of the animal; expressing the relative effect of the composition on each parameter as a numerical score; and computing the wellness related index using an algorithm that takes into account the numerical scores of the composition for the plurality of parameters.

There is also provided a marketing method that comprises communicating a wellness related index for a composition for consumption by an animal. This index can be derived by the method as described immediately above.

There is further provided a kit comprising a first ingredient and a second ingredient, wherein at least the second ingredient is associated with a wellness related index derived by the method as described above.

There is still further provided a means for communicating information about a wellness related index derived by a method of the present invention, the means being selected from product labels, package inserts, brochures, handouts, advertisements, public announcements, audiotapes, videotapes, DVDs, CD-ROMs, computer readable chips, cards or disks, computer memory, web pages, or combinations thereof.

Further features and benefits of the invention will be apparent to one skilled in the art from reading this specification.

### DETAILED DESCRIPTION OF THE INVENTION

This invention provides methods for deriving a wellness related index for a composition for consumption by an animal. The methods comprise evaluating an effect of the composition, relative to the effect of one or more comparative compositions, on each of a plurality of parameters indicative of the health or wellness of the animal; expressing the relative effect of the composition on each parameter as a numerical score; and computing the wellness related index using an algorithm that takes into account the numerical scores of the composition for the plurality of parameters.

The term “composition” means a composition nutritionally and/or medicinally adapted for consumption by an animal. Such compositions can be foods, supplements, treats, toys, pharmaceuticals or nutraceuticals. A composition suitable for a canine, for example, can illustratively be a dog food or a dog biscuit.

To evaluate an effect of a composition, relative to the effect of one or more comparative compositions, a trial is performed where each composition is administered to several animals at one time or one animal at different times. For example, when the compositions are food, then the trial is a feeding trial. Illustratively, when a group of animals are used, the animals are administered the composition or one of the comparative compositions over some period of time, for example, one or more days, weeks, or months. Alternatively, if one animal is used in the trial, the composition is administered for a duration of time, and each of the comparative compositions are administered consecutively (but not necessarily in any order) for substantially the same duration of time. At time points during the trial, a plurality of parameters indicative of the health or wellness of the animal can be measured.

The plurality of parameters measured according to the invention are related to one wellness condition or a plurality of wellness conditions. “Wellness” of an animal herein encompasses all aspects of the physical, mental, and social well-being of the animal, and is not restricted to the absence of infirmity. Wellness conditions include without limitation antioxidant status, immune status, joint health, skin condition, coat condition, cardiovascular health, kidney health, liver health, eye health, neurological health, agility, body weight, fat content, muscle content, states of parasitic infestation, sensory acuteness, dispositional and behavioral attributes, cognitive function, and other states of disease or physiological disorder. One skilled in the art will know certain parameters that relate to particular wellness conditions.

A parameter as used herein is something measured or evaluated on an animal or a sample from an animal. A sample can be, for example a tissue such as bone, muscle, etc, or can be a biofluid. The parameter can also be a measure or evaluation of an animal's actions. Some parameters can be measured several ways, and this is understood by one skilled in the art. If two or more foods are compared, then a particular parameter should be measured consistently, e.g. the same way, in order to rationally compare the foods.

In some embodiments, the plurality of parameters can comprise at least one marker obtained from an animal. The marker can be a biomarker, a biometric data point obtained from an animal, and/or a behavioral data point obtained from an animal.

Biomarkers are quantifiable clinical parameters specifically reflecting a physiological state or change in an animal in relation to health or disease. For example, a biomarker can be used to assess the presence of cancer, or a biomarker can be a chemical compound produced by a specific organism that can be used as an indicator for the presence of that organism. Suitable biomarkers can be found in, for example, biofluid or tissue samples obtained from an animal. Such biofluid or tissue samples useful herein can be any such sample that is amenable to analysis for purposes of developing the index, including biofluids or tissue samples useful for diagnostic purposes. Biofluids that can be sampled include excreta (feces and urine), blood, saliva, spinal fluid, amniotic fluid, milk, etc. Tissue samples can be obtained for example by biopsy, by surgical removal (e.g. during surgery being conducted for other purposes), by cheek swab or by pulling a few hairs.

Illustrative parameters measured from blood and/or serum include but are not limited to vitamin E, triglycerides, cholesterol, fatty acids [including omega-3 fatty acids such as eicosapentaenoic acid (EPA) and docosahexaenoic acid (DHA), omega-6 fatty acids such as linoleic acid, and others], glutathione peroxidase, alkanals, osteocalcin, cartilage oligomeric matrix protein (COMP), cross-linked amino terminal telopeptide of type I collagen (NTx), type I collagen carboxy terminal telopeptide (ICTP), taurine, blood urea nitrogen (BUN), creatinine, microalbuminuria, and combinations thereof. The term microalbuminuria relates to trace levels of albumin measured in a urine sample.

Illustrative parameters measured in urine include but are not restricted to NTx, pyrodinoline, taurine, creatinine, microalbuminuria, and combinations thereof.

In further embodiments, it can be desirable to evaluate combinations or ratios of individual parameters, for example, vitamin E to cholesterol ratio or BUN to creatinine ratio.

Illustrative parameters measured in tissue include but are not restricted to intact DNA, necrosis, hypertrophy, hyperplasia, atrophy, myopathy, inflammation, dysplasia, hypoplasia, ischemia, sclerosis, cirrhosis, and proteins or genes activated by oxidative stress.

A biometric data point is a measurable physical characteristic of an animal. Suitable biometric data points can include, but are not limited to, body weight, body fat, height, limb force, skin tone and coat condition.

A behavioral data point is a measurable action or reaction of an animal usually in relation to its environment. Suitable behavioral data points can include but are not limited to temperament, trainability and task performance.

In some embodiments, evaluating the effect of a plurality of parameters according to the invention can produce a wellness related index indicative of the effect of the composition on the overall health and wellness of an animal. Parameters that relate to the wellness related index can include but are not limited to glutathione peroxidase, vitamin E to cholesterol ratio, alkanals, osteocalcin, COMP, NTx, ICTP, pyrodinoline, EPA, DHA, total omega-3 fatty acids, taurine, BUN, creatinine, BUN to creatinine ratio, microalbuminuria, linoleic acid, intact DNA, limb force or body weight.

A large number of parameters can be used in computing a particular index. Alternatively, fewer parameters, or a subset of parameters, can be used. Such a subset can still, in many cases, accurately reflect the effect of a composition. Measuring a subset of parameters, as opposed to many parameters, may be desirable to save time and cost. In some embodiments, a minimum set of parameters is evaluated consistent with providing an index that is an accurate reflection of the composition.

In other embodiments, selection of a subset of parameters relevant to a particular life stage of the animal can produce an index indicative of the effect of the composition on the health and wellness of the animal during that life stage. For example, a healthy growth index is indicative of the effect of the composition on the health and wellness of a young animal such as a puppy or kitten, a healthy adult maintenance index is indicative of the effect of the composition on the health and wellness of an adult animal, and a healthy aging index is indicative of the effect of the composition on the health and wellness of a senior or super senior animal. As used herein, “senior” or “super senior” refer to life-stages of an animal. Illustratively, for small or regular breed canines, the senior life stage is from about 7 to about 10 years of age. For felines, the senior life stage is from about 7 to about 12 years of age. For small or regular breed canines, the super senior life stage is any age greater than 10 years of age. For large breed canines, the senior and super senior life stage is any age greater than 5 years of age. For felines, the super senior life stage is any age greater than 12 years of age.

In other embodiments, the selection of a subset of parameters relevant to the aging process, for example, vitamin E to cholesterol ratio, COMP, NTx, ICTP, taurine, BUN, microalbuminuria and body weight can produce a healthy aging index. The healthy aging index is indicative of the effect of the composition on the health and wellness of an aging animal. This healthy aging index can be tailored to a specific animal. For example, when the animal is a canine, the parameters contributing to the healthy aging index can include vitamin E to cholesterol ratio, COMP, NTx, taurine, BUN, microalbuminuria, and body weight. When the animal is a feline, the parameters contributing to the healthy aging index can include vitamin E to cholesterol ratio, NTx, ICTP, BUN, microalbuminuria and body weight. In addition, parameters may also include a measure of the amount of weight a senior or supersenior animal places on an arthritic limb. Said “force/limb” parameter can be measured using conventional methods, e.g., employing force plates familiar to one of skill in the art.

In some embodiments, selection of a subset of parameters relating to joint health and/or agility can produce a mobility index. This subset of parameters can include, for example, osteocalcin, COMP, NTx, EPA, limb force and body weight.

In other embodiments, selection of a subset of parameters relating to neurological health can produce a cognitive index. This subset of parameters can include, for example, glutathione peroxidase, vitamin E to cholesterol ratio, DHA, intact DNA (measured by a technique such as a comet assay) and alkanals.

In still other embodiments, selection of a subset of parameters relating to prevention and/or treatment of one or more diseases can produce a disease prevention index. An example of such an index can be a kidney health index and the subset of parameters can include, for example, vitamin E to cholesterol ratio, EPA, BUN and urine microalbuminuria.

It is contemplated herein that one of skill in the art is capable of evaluating the effect of a composition on a particular parameter and assessing the overall positive or negative effect of the composition on said parameter. For example, it is desirable for a food composition to increase glutathione peroxidase levels in an animal as the role of this family of enzymes is to protect an organism from oxidative damage. In contrast, high levels of alkanals are not desired as these compounds typically are products of oxidative stress. Compositions which can modify levels of various parameters, e.g., increase or decrease said levels, as the case may be (see for example, Table 6), may then be compared and ranked to derive a wellness related index as disclosed herein.

Subsequent to evaluating the effect of the composition and the one or more comparative compositions on a plurality of parameters, the relative effect of the composition on each parameter is expressed as a numerical score. In one embodiment, the numerical score for each parameter correlates positively to a desired wellness effect of the parameter. The numerical score is a number related to the absolute value, i.e. the level of a biomarker or the weight of the animal. This number can be positively or negatively correlated with the parameter, but in general a higher value is more closely indicative of an ideal score. The numerical score can be an integer or non-integer, the scores can be consecutive or non-consecutive. The numerical score for each parameter can be an unweighted indicator of position of the composition in a ranking of the composition and comparative compositions with respect to effect on the parameter. The ranking can be made up of a series of any numbers or a series of integers.

A wellness related index is computed using an algorithm that takes into account the numerical scores of the composition for the plurality of parameters. The algorithm can take into account the numerical scores for all the parameters or a selection of the parameters. The algorithm can be a simple arithmetic summary or total of scores, expressed as a fraction, e.g. a percentage, of the maximum possible score. The numerical scores included in this computation can be unweighted, or the scores can be weighted according to the individual significance of each parameter as a wellness indicator.

In some embodiments, the numerical score for each parameter correlates positively to a desired wellness effect of the parameter, and the algorithm sums the numerical scores and expresses the resulting total score for the composition as a fraction or percentage of the maximum possible score.

In a further embodiment, the numerical score for each parameter is an unweighted indicator of position of the composition in a ranking of the composition and comparative compositions with respect to effect on the parameter. The numerical score can be one of a series of consecutive or non-consecutive numerical integers. In a preferred embodiment, the numerical score is one of a series of consecutive numerical integers (including a fraction thereof if there is a tie score).

The wellness related index can then be used to compare different compositions, providing an easily visualized method of determining the composition most suitable for an animal, and indicates a composition's effect on health and wellness in that animal.

In some embodiments, the algorithm can further compute a measure of statistical confidence in the index, for example, a P-value.

Compositions discussed herein can be selected for any of a variety of animals, for example, the animal can be human or non-human. In various embodiments, the animal is a vertebrate, for example a fish, a bird, a reptile or a mammal. Illustratively among mammals, the animal can be a member of the order Carnivora, including without limitation canine and feline animals.

In a particular embodiment, the animal is a companion animal. A “companion animal” herein is an individual animal kept by a human caregiver as a pet, or any individual animal that has been widely domesticated as pets, including dogs (Canis familiaris) and cats (Felis domesticus), whether or not the individual animal is kept solely or partly for companionship. Thus “companion animals” herein include working dogs, farm cats kept for rodent control, etc., as well as pet dogs and cats.

In various embodiments, compositions discussed herein can be useful in nutrition of domesticated animals including livestock (e.g., porcine, equine, ovine, bovine and caprine), laboratory animals (e.g., murines including rats and mice; and non-human primates such as monkeys, chimpanzees, etc.), companion and pet animals (e.g., dogs, cats, hamsters, guinea pigs, gerbils, rabbits, ferrets, chinchillas, etc.), avians (e.g., domestic birds, such as canaries, parrots, etc. and commercial gallinaceous animals such as chickens, ducks, geese, turkeys, etc.).

There is provided a marketing method comprising communicating a wellness related index, for example, derived by the method as described above, for a composition for consumption by an animal. In some embodiments, the communicating is accomplished by any communicating means known in the art such as product labels, package inserts, brochures, handouts, advertisements, public announcements, audiotapes, videotapes, DVDs, CD-ROMs, computer readable chips, cards and disks, computer memory, web pages and combinations thereof.

There is also provided a kit comprising a first ingredient and a second ingredient, wherein at least the second ingredient is associated with a wellness related index derived by the method as described above. In one embodiment, the first ingredient is a base food and the second ingredient is a nutritional supplement. In another embodiment, the first ingredient and the second ingredient are associated with a wellness related index. In some embodiments, the kit further comprises instructions for one or more of (1) preparing a composition with a wellness related index derived by the method as described above by combining the first ingredient and the second ingredient and, optionally, additional ingredients that are not part of the kit, and (2) feeding the composition to an animal. The kit can further comprise a means of communicating information relating to the wellness related index. The communicating means can be, for example, a label or package insert.

In some embodiments, the ingredients are presented in separate containers, which can be co-packaged or distributed in separate packages. In other embodiments, the ingredients are presented in a virtual package. The term “virtual package” relates to a kit wherein the separate components are not physically packaged together but where directions exist, for example on a product label, advertisement, etc., instructing the user to bring those components together in a way that provides a composition with a wellness related index derived by, for example, the method as described above. This can include, for example, a bag containing one ingredient and directions instructing the user to go to a website, contact a recorded message, view a visual message, or contact a caregiver to obtain instructions on how to use the kit.

There is still further provided a means for communicating information about a wellness related index derived by the method as described above for a composition for consumption by an animal. The means comprises a document, digital storage media, optical storage media, audio presentation, or visual display containing the information or instructions. In certain embodiments, the communication means is a displayed web site, visual display, brochure, product label, package insert, advertisement, handout, public announcement, audiotape, videotape, DVD, CD-ROM, computer readable chip, computer readable card, computer readable disk, computer memory, or combination thereof containing such information or instructions.

This means for communicating information about a wellness related index can be attached to, enclosed in, or separate from an article of manufacture comprising a package containing an animal food subject to the index. Any form of package appropriate to the nature of the composition can be used, including without limitation a can, a jar, a tube, a pouch, a bag, a bag in a box, etc.

This invention also provides a method for comparing two or more compositions for consumption by an animal. The method comprises evaluating the effect of each composition on each of a plurality of wellness related parameters in an animal consuming the composition; ranking the compositions according to the effect, to provide a numerical score for each parameter in each composition; and computing a single numerical index for a composition according to an algorithm that takes into account the numerical scores of the composition for the plurality of parameters. This method can be performed as described above for deriving a wellness related index.

### EXAMPLES

The following examples are merely illustrative, and not limiting to this disclosure in any way. Abbreviations used in the following examples include:

| | |
|---|---|
| COMP | cartilage oligomeric matrix protein |
| NTx | cross linked amino-terminal telopeptide of Type I collagen |
| ICTP | Type I collagen carboxy-terminal telopeptide |
| EPA | eicosapentaenoic acid |
| DHA | docosahexaenoic acid |
| BUN | blood urea nitrogen. |

Statistical analysis is discussed with respect to the examples herein, however it will be recognized that significant differences between wellness indexes are not a requirement of the present invention.

### Example 1

Prior to the feeding study, 40 dogs were given the same dog food containing sufficient protein, fat, fatty acids and vitamins to meet minimum AAFCO requirements. The dogs were over 10 years of age. Upon beginning the study, the dogs were divided into 4 groups of 10 dogs each. Each group was assigned to one of four dog foods as shown in Table 1.

The dogs were fed the assigned food for 90 days. Urine and blood samples were collected at day 0, 30, and 90. Vitamin E, triglyceride, cholesterol and fatty acid levels were measured in blood serum. Other biomarkers were measured in urine or as biometric data. Table 1 is an example of a Healthy Aging Index determined by evaluating each food according to its effect on each of the parameters, then expressing the relative effect of that food as a numerical score. For example, when glutathione peroxidase was measured, Food 4 ranked the highest and was given a numerical score of “4” for that parameter. Food 3, on the other hand, received a numerical score of “1” for the same parameter as it ranked the lowest of the four foods. When creatinine was measured, Food 1 received a numerical score of “1” and Food 2 received a score of “4”, while Foods 3 and 4 had an equal effect on creatinine intermediate between Foods 1 and 2, and thus each received a score of “2.5”. The numerical scores obtained for all the parameters were totaled for each food, and then further expressed as a percent of the total possible score. Table 1 shows that out of a possible 68 points, the Food 1 scored 60 points (88%). This score was the highest score obtained by the 4 foods tested, indicating that Food 1 had a more desirable effect on parameters measured that relate to aging. The percentages obtained represent the healthy aging index of the respective composition.

Statistical confidence was determined using the T-test. When compared to the Food 1, each of the other three foods was statistically different as demonstrated by their respective P-values.

TABLE 1
Healthy Aging Index of Four Dog Foods

Score

| Parameter | Food 1 | Food 2 | Food 3 | Food 4 |
|---|---|---|---|---|
| Glutathione | 3 | 2 | 1 | 4 |
| peroxidase | | | | |
| Vitamin E: | 3 | 2 | 4 | 1 |
| cholesterol | | | | |
| Bone specific | 3 | 2 | 4 | 1 |
| alkaline | | | | |
| phosphatases | | | | |
| Osteocalcin | 4 | 3 | 2 | 1 |
| COMP | 4 | 3 | 2 | 1 |
| NTx | 3 | 1 | 2 | 4 |
| EPA | 4 | 3 | 1 | 2 |
| DHA | 4 | 2 | 1 | 3 |
| Total ω-3 Fatty | 4 | 2 | 1 | 3 |
| Acids | | | | |
| Taurine | 4 | 3 | 2 | 1 |
| BUN: | 4 | 2 | 3 | 1 |
| creatinine | | | | |
| BUN | 4 | 2 | 3 | 1 |
| Creatinine | 1 | 4 | 2.5 | 2.5 |
| Urine micro- | 4 | 2 | 3 | 1 |
| albuminuria | | | | |
| Linoleic acid | 4 | 3 | 1 | 2 |
| Force/limb | 4 | 3 | 1 | 2 |
| Body weight | 3 | 4 | 1 | 2 |
| Total score | 60 | 43 | 34.5 | 32.5 |
| T-test, P-value | | 0.0052 | 0.0007 | 0.0004 |
| Total possible | 68 | 68 | 68 | 68 |
| points | | | | |
| % of total | 88% | 63% | 51% | 48% |

Table 2 shows the same data as Table 1, however only data for 7 parameters are included. The Food 1 scored 25 out of the possible 28 points (89%), the highest score relative to the other 3 foods tested. The P-values show that even when the number of parameters measured was limited to 7, the differences between Food 1 scores and the respective scores of the other foods were statistically significant. Thus, using a subset of relevant parameters can still provide indexes that distinguish between foods.

TABLE 2
Healthy Aging Index of Four Dog Foods with Minimum Parameters

Score

| Parameter | Food 1 | Food 2 | Food 3 | Food 4 |
|---|---|---|---|---|
| Vitamin E: | 3 | 2 | 4 | 1 |
| cholesterol | | | | |
| COMP | 4 | 3 | 2 | 1 |
| NTx | 3 | 1 | 2 | 4 |
| Taurine | 4 | 3 | 2 | 1 |
| BUN | 4 | 2 | 3 | 1 |
| Urine micro- | 4 | 2 | 3 | 1 |
| albuminuria | | | | |
| Body weight | 3 | 4 | 1 | 2 |
| Total | 25 | 17 | 17 | 11 |
| T-test, P-value | | 0.03002 | 0.03002 | 0.01340 |
| Total possible | 28 | 28 | 28 | 28 |
| points | | | | |
| % of total | 89% | 61% | 61% | 39% |

Table 3 shows data collected in Example 1. Parameters measured were selected for their pertinence to one of three indexes: cognitive function, mobility, and kidney health. Foods were ranked according to their effect on each parameter as described herein. These data show that the present invention is useful for establishing a variety of indexes, including cognitive function, mobility, and kidney health indexes.

TABLE 3
Cognitive Function, Mobility, and Kidney Health Indexes

| | | | | | | |
|---|---|---|---|---|---|---|
| Parameter | Technical claim | Food 1 | Food 2 | Food 3 | Food 4 | Total Points |

COGNITIVE FUNCTION INDEX - CANINE

| | | | | | | |
|---|---|---|---|---|---|---|
| Glutathione | Antioxidant | 3 | 2 | 1 | 4 | 10 |
| peroxidase | status | | | | | |
| Vitamin E: | Antioxidant | 3 | 2 | 4 | 1 | 10 |
| Cholesterol | status | | | | | |
| DHA | Antioxidant | 4 | 2 | 1 | 3 | 10 |
| | status | | | | | |
| Comet assay, % | Antioxidant | 4 | 2.5 | 1 | 2.5 | 10 |
| intact DNA | status | | | | | |
| Alkanals | Antioxidant | 3 | 2 | 4 | 1 | 10 |
| | status | | | | | |
| Total score | | 17 | 10.5 | 11 | 11.5 | |
| T-test, P-value | | | 0.0029 | 0.2606 | 0.1194 | |
| Total possible points | | 20 | 20 | 20 | 20 | |
| % of total | | 85% | 53% | 55% | 58% | |

MOBILITY INDEX - CANINE

| | | | | | | |
|---|---|---|---|---|---|---|
| Osteocalcin | Joint health | 4 | 3 | 2 | 1 | 10 |
| COMP | Joint health | 4 | 3 | 2 | 1 | 10 |
| NTx | Joint health | 3 | 1 | 2 | 4 | 10 |
| EPA | Joint health | 4 | 3 | 1 | 2 | 10 |
| Force/limb | Joint health | 4 | 3 | 1 | 2 | 10 |
| Body weight | Joint health | 3 | 4 | 1 | 2 | 10 |
| | maintain weight | | | | | |
| Total score | | 22 | 17 | 9 | 12 | |
| T-test, P-value | | | 0.0925 | 0.0009 | 0.0422 | |
| Total possible points | | 28 | 28 | 28 | 28 | |
| % of total | | 79% | 61% | 32% | 43% | |

KIDNEY HEALTH INDEX - CANINE

| | | | | | | |
|---|---|---|---|---|---|---|
| Vit E: | Kidney health | 3 | 2 | 4 | 1 | 10 |
| Cholesterol | | | | | | |
| EPA | Kidney health | 4 | 3 | 1 | 2 | 10 |
| BUN | Kidney health | 4 | 2 | 3 | 1 | 10 |
| Urine micro- | Kidney health | 4 | 2 | 3 | 1 | 10 |
| albumnuria | | | | | | |
| Total score | | 15 | 9 | 11 | 5 | |
| T-test, P-value | | | 0.0138 | 0.3081 | 0.0032 | |
| Total possible points | | 20 | 20 | 20 | 20 | |
| % of total | | 75% | 45% | 55% | 25% | |

### Example 2

Prior to the feeding study, 40 cats were given the same cat food containing sufficient protein, fat, fatty acids and vitamins to meet minimum AAFCO requirements. Upon beginning the study, the cats were divided into 4 groups of 10 cats each. The cats were over 12 years of age. Each group was assigned to one of four cat foods.

Cats were fed the assigned food for 90 days. Urine and blood samples were collected at day 0, 30, and 90 and various parameters were measured as described in Example 1. Table 4 is an example of a Healthy Aging Index determined by evaluating each food according to its effect on each of the parameters, then expressing the relative effect of that food as a numerical score. For example, when vitamin E to cholesterol ratio was measured, Food 1 and Food 4 received a numerical score of “3.5” as both foods shared the highest ranking for that parameter. Food 3 received a numerical score of “1” indicating the lowest ranking for vitamin E to cholesterol ratio. The numerical scores obtained for all the parameters were totaled for each food, and then further expressed as a percent of the total possible score. Table 4 shows that out of a possible 64 points, the Food 1 scored 53 points (83%). This score was the highest score obtained by the 4 foods tested. The percentages obtained represent the healthy aging index of the respective composition.

Statistical confidence was determined using the T-test. When compared to Food 1, Food 2 and Food 4 were statistically different as demonstrated by their respective P-values.

TABLE 4
Healthy Aging Index of Four Cat Foods

Score

| Parameter | Food 1 | Food 2 | Food 3 | Food 4 |
|---|---|---|---|---|
| Glutathione | 4 | 2 | 1 | 3 |
| peroxidase | | | | |
| Vitamin E: | 35 | 2 | 1 | 3.5 |
| cholesterol | | | | |
| Bone specific | 2 | 1 | 3 | 4 |
| alkaline | | | | |
| phosphatases | | | | |
| NTx | 4 | 3 | 1 | 2 |
| Pyrodenoline | 3 | 2 | 4 | 1 |
| ICTP | 4 | 2 | 1 | 3 |
| EPA | 4 | 1 | 3 | 2 |
| DHA | 4 | 2 | 1 | 3 |
| Total ω-3 Fatty | 4 | 1 | 3 | 2 |
| Acids | | | | |
| Taurine | 1 | 2 | 4 | 3 |
| BUN | 4 | 2 | 3 | 1 |
| Creatinine | 4 | 1 | 3 | 2 |
| Urine micro- | 4 | 1 | 2.5 | 2.5 |
| albuminuria | | | | |
| Linoleic acid | 3 | 2 | 4 | 1 |
| Force/limb | 1 | 4 | 2 | 3 |
| Body weight | 2.5 | 2.5 | 2.5 | 2.5 |
| Total score | 53 | 30.5 | 40 | 36.5 |
| T-test, P-value | | 0.0015 | 0.0981 | 0.0109 |
| Total possible | 68 | 68 | 68 | 68 |
| points | | | | |
| % of total | 82% | 46% | 60% | 61% |

Table 5 shows the same data as Table 4, however only data for 6 parameters are included. Food 1 scored 22 out of the possible 24 points (92%), the highest score relative to the other 3 foods tested. The P-values show that even when the number of parameters measured was limited to 7, the differences between Food 1 score and the respective scores of the other foods were statistically significant. Again, using a subset of relevant parameters can still provide indexes that distinguish between foods.

TABLE 5
Healthy Aging Index of Four Cat Foods with Minimum Parameters

Score

| Parameter | Food 1 | Food 2 | Food 3 | Food 4 |
|---|---|---|---|---|
| Vitamin E: | 3.5 | 2 | 1 | 3.5 |
| cholesterol | | | | |
| NTx | 4 | 3 | 1 | 2 |
| ICTP | 4 | 2 | 1 | 3 |
| BUN | 4 | 2 | 3 | 1 |
| Urine micro- | 4 | 1 | 2.5 | 2.5 |
| albuminuria | | | | |
| Body weight | 2.5 | 2.5 | 2.5 | 2.5 |
| Total score | 22 | 12.5 | 11 | 14.5 |
| T-test, P-value | | 0.0126 | 0.0139 | 0.0476 |
| Total possible | 24 | 24 | 24 | 24 |
| points | | | | |
| % of total | 92% | 52% | 46% | 60% |

TABLE 6
Generally Desired Effect of a Composition on Various Parameters

| Parameter | Effect (on level or ratio) |
|---|---|
| Glutathione peroxidase | increase |
| Vitamin E cholesterol | increase |
| Bone specific alkaline | decrease |
| phosphatases | |
| Osteocalcin | increase |
| COMP | decrease |
| NTx | decrease |
| EPA | increase |
| DHA | increase |
| Total ω-3 Fatty Acids | increase |
| Taurine | increase |
| BUN : creatinine | decrease |
| BUN | decrease |
| Creatinine | decrease |
| Urine micro-albuminuria | decrease |
| Linoleic acid | increase |
| Force/limb | increase |
| Body weight | maintain |
| Comet assay, % intact DNA | increase |
| Alkanals | decrease |
| Pyrodenoline | decrease |
| ICTP | decrease |

This invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise. Similarly, the words “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents, patent applications, and publications mentioned herein are incorporated herein by reference to the extent allowed by law for the purpose of describing and disclosing the compounds, processes, techniques, procedures, technology, articles, and other compositions and methods disclosed therein that might be used with the present invention. However, nothing herein is to be construed as an admission that the invention is not entitled to antedate such disclosure by virtue of prior invention.

What is claimed is:

1. A method for deriving a wellness related index for a composition for consumption by an animal comprising:
(a) evaluating an effect of the composition, relative to the effect of one or more comparative compositions, on each of a plurality of parameters indicative of the health or wellness of the animal;
(b) expressing the relative effect of the composition on each parameter as a numerical score; and
(c) computing the wellness related index using an algorithm that takes into account the numerical scores of the composition for the plurality of parameters.

2. The method of claim 1 wherein the numerical score for each parameter correlates positively to a desired wellness effect on the parameter; and the algorithm comprises summing the numerical scores and expressing the resulting total score for the composition as a fraction or percentage of the maximum possible total score.

3. The method of claim 2 wherein the numerical score for each parameter is an unweighted indicator of position of the composition in a ranking of the composition and comparative compositions with respect to effect on the parameter.

4. The method of claim 1 wherein the numerical score is one of a series of consecutive numerical integers.

5. The method of claim 1 wherein the animal is canine or feline.

6. The method of claim 1 wherein the plurality of parameters comprise at least one biomarker obtained from the animal.

7. The method of claim 1 wherein the parameters evaluated are each related to one or more wellness conditions selected from the group consisting of antioxidant status, immune status, joint health, skin condition, coat condition, cardiovascular health, kidney health, liver health, eye health, neurological health, agility, body weight, fat content, muscle content, states of parasitic infestation, sensory acuteness, dispositional and behavioral attributes and cognitive function.

8. The method of claim 1 wherein the parameters evaluated comprise parameters selected from the group consisting of glutathione peroxidase, vitamin E to cholesterol ratio, alkanals, osteocalcin, cartilage oligomeric matrix protein, NTx, ICTP, pyrodinoline, eicosapentaenoic acid, docosahexaenoic acid, total omega-3 fatty acids, taurine, blood urea nitrogen, creatinine, blood urea nitrogen to creatinine ratio, microalbuminuria, linoleic acid, intact DNA, limb force and body weight.

9. The method of claim 1 wherein the parameters relate to joint health and/or agility and the resulting index is a mobility index.

10. The method of claim 1 wherein the parameters relate to neurological health and the resulting index is a cognitive index.

11. The method of claim 1 wherein the animal is senior or super senior and the resulting index is a healthy aging index.

12. The method of claim 11 wherein the parameters contributing to the healthy aging index comprise individual parameters selected from the group consisting of vitamin E to cholesterol ratio, cartilage oligomeric matrix protein, NTx, ICTP, blood urea nitrogen, taurine, microalbuminuria, and body weight.

13. The method of claim 11 wherein the animal is canine and the parameters contributing to the healthy aging index comprise individual parameters selected from the group consisting of vitamin E to cholesterol ratio, cartilage oligomeric matrix protein, NTx, taurine, blood urea nitrogen, microalbuminuria, and body weight.

14. The method of claim 11 wherein the animal is feline and the parameters contributing to the healthy aging index comprise individual parameters selected from the group consisting of vitamin E to cholesterol ratio, NTx, ICTP, blood urea nitrogen, microalbuminuria, and body weight.

15. The method of claim 1 wherein the animal is a juvenile or an adult and the resulting index is a healthy adult maintenance index.

16. The method of claim 1 wherein the compositions are independently selected from the group consisting of foods, supplements, treats, toys, pharmaceuticals and nutraceuticals.

17. A marketing method comprising communicating a wellness related index, derived by the method of claim 1, for a composition for consumption by an animal.

18. The method of claim 17 wherein the communicating is accomplished by a communicating means selected from the group consisting of product labels, package inserts, brochures, handouts, advertisements, public announcements, audiotapes, videotapes, DVDs, CD-ROMs, computer readable chips, cards and disks, computer memory, web pages, and combinations thereof.

19. A means for communicating information about a wellness related index derived by the method of claim 1 for a composition for consumption by an animal comprising a document, digital storage media, optical storage media, audio presentation, or visual display containing the information or instructions.

20. The means of claim 19 selected from the group consisting of product labels, package inserts, brochures, handouts, advertisements, public announcements, audiotapes, videotapes, DVDs, CD-ROMs, computer readable chips, cards and disks, computer memory, web pages, and combinations thereof.
